(19) United States
(12) Patent Application Publication
(10) Pub. No.: US 2018/0178970 A1
(43) Pub. Date: Jun. 28, 2018

(54) ACTUATED-VALVE METERING

(71) Applicant: The Technology Partnership Plc, Royston, Hertfordshire (GB)

(72) Inventors: Daniel STRANGE, Royston, Hertfordshire (GB); Robert SELBY, Royston, Hertfordshire (GB); William RICHARDSON, Royston, Hertfordshire (GB)

(21) Appl. No.: 15/736,522

(22) PCT Filed: Jun. 15, 2016
(86) PCT No.: PCT/GB2016/051781

§ 371 Date: Dec. 14, 2017

(30) Foreign Application Priority Data
Jun. 15, 2015 (GB) 1510451.6

(51) Int. Cl. B65D 83/54; G01F 11/02; B65D 83/14

(52) U.S. Cl. CPC B65D 83/54; A61M 11/007; B65D 83/75; G01F 11/021

(57) ABSTRACT

According to a first aspect of the disclosure, a spray device is provided for generating an aerosol. The spray device comprises a reservoir for containing a store of a liquid; a delivery chamber for containing a dose of the liquid received from the reservoir prior to ejection; a perforate element comprising one or more nozzles; a drive mechanism configured to drive liquid from the delivery chamber through the one or more nozzles; and a fluid metering system. The fluid metering system comprises an actuated valve that is actuatable between a closed position and an open position. The opening and closing of the actuated valve can be sequenced to control the flow of fluid from the reservoir to the delivery chamber.

### Related Applications

The present application is a national stage application under 35 U.S.C. § 371 of International Application No. PCT/GB2016/051781, filed 15 Jun. 2016, which claims priority to Great Britain Patent Application No. 1510451.6, filed 15 Jun. 2015. The above referenced applications are hereby incorporated by reference into the present application in their entirety.

### FIELD OF INVENTION

The present invention relates to spray devices for generating aerosols. Specifically, the present invention relates to spray devices in which the pressure profile of fluid to be aerosolised has a short start-up time and decay time at the beginning and end of a spray event.

### BACKGROUND OF THE INVENTION

Aerosol systems that can generate a slow moving mist, such as those described in PCT/GB2015/051413 and GB1420266.7 are highly effective and user-friendly methods of delivering pharmaceutical ingredients to the lungs, nose, eyes, and skin. The mist is monodisperse and is much more controllable than a typical pump spray. This enables delivery to be targeted, with fast uptake and an avoidance of undesirable effects, such as over spray in the case of nasal delivery or poorly controlled droplet distribution at the start up and end of the droplet delivery.

Such systems operate by forcing liquid at high pressures through micro-structured nozzles containing orifices with hole sizes of 100 μm or less. The pressure is often generated by use of a compression spring that acts on a piston to generate a substantially constant pressure. The compression spring is often primed by the user of the device.

When a constant pressure is applied to the fluid path within a typical spray pump, the pressure compresses any compliant seals and any trapped air bubbles, while at the same time forcing liquid through the nozzle. When the force is removed from the system, the compressed seals and air continue to apply a pressure to the liquid that gradually decays with time. The residual pressure can continue to force liquid through the nozzle for several seconds after the force is removed. This system compliance can adversely affect the performance of the spray, especially during the start-up and end of a spray delivery event. There is a need, therefore, for a means of controlling the pressure profile in the liquid at the start and end of the delivery event, in order to produce a fast start and a fast decay of the pressure in the liquid in response to the application and removal of a force input.

In such Aerosol systems, it is often desirable and necessary to meter the delivery to deliver a given fluid dose volume, the metering system should be low cost, particularly for the consumer healthcare market where cost of goods for delivery systems is a major consideration. Keeping costs well below $1 and more favourable below $0.2 for the pump spray components is key to achieving a commercially competitive device. For pressurised aerosols, a rapid rise in fluid pressure at the beginning of a delivery event, a fast start, is beneficial for creating an aerosol that is consistent in terms of plume shape and droplet size distribution. Similarly, it is beneficial to have a rapid decay in fluid pressure at the end of a delivery event, a fast end, rather than a gradual reduction in pressure. The response of the compliance within the fluid path , metering system, seals and piston, to the application or removal of pressure is often the limiting factor in the time constant of the fluid pressure. Achieving a sharp response at the start and end of the spray by providing a highly non-compliant fluid path can be challenging with low cost materials, and so an additional control to the pressure within the fluid chamber is beneficial.

GB application GB 2 251 898 A provides a method of achieving a fast start in which a pre-loaded valve, termed a pressure relieving valve, is used to prevent aerosolising of fluid before the pressure in a metered chamber exceeds a particular threshold value. While this approach has some benefits, it can be difficult to achieve a desired pressure consistently from a small valve.

For medical drug delivery applications where devices are typically only used relatively few times before being disposed, there is a need for low cost devices. This need for low cost is even more important for over the counter drug delivery devices where there is further pressure on cost of goods. By designing multiple functions into moulded plastic components several costly factors can be reduced, including the part count, and assembly cost; therefore, the overall cost of the device can be minimised. The solutions described in this application eliminate multiple small valve components and thereby reduce the part count and hence cost of goods.

The application of metering methods to collision based droplet formation methods has been described in GB1420266.7 where impinging jets are used to induce break-up into small droplets and PCT/GB2015/051413 where an impact surface induces break up of a liquid jet. This invention is applicable to both of these droplet delivery technologies and can also be applied to droplet generation and delivery devices where droplet formation is by induced jet instability.

Rayleigh break up of a jet stream relies on the propagation of the most favourable frequency of system noise such that the instabilities in the jet grow until they dissect the jet. Droplets are formed as the jet breaks up. This means can be used to form droplets from a pressurised spray but can prove problematic due to long jet length required for the instabilities to grow to a sufficient magnitude to cause the jet to break-up. Inconsistencies in the break-up length and droplet size can be caused by significant variations in the noise and instability propagation. Inducing additional instability in the jet through the use of non-circular jets formed from non-circular nozzles can reduce the break-up distance. A non-circular jet has a greater surface area for a given mass flow than a circular jet. This additional energy drives the growth of instability in the jet. In experiments with a nozzle with approximately a 2:1 ratio between the minimum and maximum diameters and a maximum dimension of approximately 13 microns operating at 150 bar where the jet speed can be approximately related to the operating pressure by the expression

operating pressure=½pv²

Droplets of 30 microns average diameter can be formed with break up occurring within a few millimetres of the exit of the nozzle by using a jet speed of approximately 170 m/s. Operating at lower pressures the break up takes longer and may need more extreme non-circularity of nozzle to maintain short and reliable break-up of the jet.

### SUMMARY OF THE INVENTION

The present disclosure includes examples of metering systems where the swept volume of an actuating piston defines the delivered dose and the refilling of the delivery chamber is achieved by either an actuated valve or by replacing a dose unit component. One aim of these metering systems are to provide a low part count and hence low cost solution to metering doses of medicament without the need for micro-machined parts or complicated assembly procedures.

These examples of metering systems are combined with an actuator and delivery chamber that provides a fast start to the aerosolisation process.

The present invention uses an actuated valve where the actuated valve's state is coupled to the condition of the device directly, so that the timing of metering and dose delivery can be made directly. Another approach to achieving a fast start is afforded by use of an actuator which has an acceleration gap such that on first release the actuator gains some momentum and kinetic energy that can be used to boost the initial pressurisation of the system to reach the preferred operating pressure quickly. This mechanism is further adapted to provide a mechanical stop so that actuator is mechanically unloaded at the end of the dose and so provides a rapid pressure reduction and clean end of the aerosolisation process.

A further benefit of the use of actuated valves is that at the end of the delivery the valve can be opened briefly to discharge residual pressure quickly and prevent slow pressure decay.

According to a first aspect of the disclosure, a spray device is provided for generating an aerosol, the spray device comprising: a reservoir for containing a store of a liquid; a delivery chamber for containing a dose of the liquid received from the reservoir prior to ejection; a perforate element comprising one or more nozzles; a drive mechanism configured to drive liquid from the delivery chamber through the one or more nozzles, thereby forming a liquid spray having one or more streams of liquid; and a fluid metering system, the fluid metering system comprising an actuated valve that is actuatable between a closed position and an open position; wherein the reservoir and the delivery chamber are in fluid communication when the actuated valve is open, and the reservoir and the delivery chamber are not in fluid communication when the actuated valve is closed, and wherein the opening and closing of the actuated valve can be sequenced to control the flow of fluid from the reservoir to the delivery chamber.

In some examples, the actuated valve comprises a moveable sleeve.

In some examples, the actuated valve comprises a body wall disposed between the reservoir and the delivery chamber.

In some examples, the moveable sleeve is disposed between the body wall and the delivery chamber.

In some examples, wherein the moveable sleeve is moveable with respect to the body wall.

In some examples, the actuated valve is moveable between an open position and a closed position by a rotary motion of the moveable sleeve.

In some examples, the actuated valve is moveable between the open position and the closed position by an axial translation of the moveable sleeve.

In some examples, the moveable sleeve comprises at least one fluid transfer port.

In some examples, the body wall comprises at least one fluid transfer port.

In some examples, the actuated valve is in the open position when at least one fluid transfer port of the body wall is aligned with at least one fluid transfer port of the moveable sleeve.

In some examples, the reservoir comprises an outer body, the outer body comprising: a first portion having approximately the same radius as the moveable sleeve, and a second portion having a greater radius than the moveable sleeve, and wherein the moveable sleeve is disposed within the reservoir with a portion of the moveable sleeve being disposed within the first portion of the outer body, such that, in use, the liquid in the reservoir is held in contact with the second portion of the outer body and a portion of the sleeve that is not disposed within the first portion.

In some examples, the moveable sleeve comprises at least one fluid transfer port; and wherein the actuated valve is closed when the moveable sleeve is in in a first position and wherein the actuated valve is open when the moveable sleeve is in a second position, wherein: in the first position the fluid transfer port is positioned adjacent to the first portion of the outer body, and in the second position, the fluid transfer port is positioned such that it is surrounded by the second portion of the outer body, such that, in use, the liquid in the reservoir is prevented from passing from the into the delivery chamber when the moveable sleeve is in the first position, and the liquid in the reservoir is able to pass from the reservoir into delivery chamber through the at least one fluid transfer valve when the moveable sleeve is in the second position.

In some examples, the first portion outer body comprises a plurality of channels that extend from the reservoir into the first portion, and wherein the actuated valve is closed when the moveable sleeve is in a first position wherein the channels are covered by the moveable sleeve, and the actuated valve is open when the moveable sleeve is in a second position wherein the channels are partially uncovered by moveable sleeve, with the channels forming a path between the reservoir and the delivery chamber.

In some examples, the spray device further comprises: a seal disposed between the first portion of the outer body and the moveable sleeve for preventing the liquid in the reservoir from flowing between the first portion of the outer body and the moveable sleeve.

In some examples, the spray device further comprises: a collision means comprising at least one impaction surface located downstream of the one of more nozzles, such that, in use, the liquid that is driven through the one or more nozzles impacts the impaction surface of the collision means.

In some examples, the one or more nozzles comprise: at least two opposing nozzles, the opposing nozzles being positioned such that the projected areas of the holes at least partially intersect at the outlet side of the perforate element such that, in use, an aerosol is generated from at least two impinging jets formed when liquid is driven through the one or more nozzles.

In some examples, the spray device is configured such that the liquid that is driven through the nozzles forms a jet stream that breaks up into droplets by propagation of instability in the jet stream.

In some examples, the drive mechanism comprises a piston.

In some examples, the drive mechanism further, comprises a drive member, the drive member being coupled to a force generating means, wherein the spray device is configured such that upon triggering the drive mechanism, the force generating means accelerates the drive member across an acceleration gap before engaging the piston, after which the drive mechanism continues to drive the piston into the delivery chamber in order to drive the liquid from the delivery chamber through the one or more nozzles.

In some examples, the force generating means comprises a spring.

In some examples, the force generating means comprises a compressed gas.

In some examples, the spray device of any preceding claim, further comprises: an actuator configured to move the actuated valve between the open position and the closed position.

In some examples, each nozzle comprises an inlet with a hydraulic diameter of 5 μm to 100 μm (typically 30 μm) and an outlet with a hydraulic diameter of 5 μm to 100 μm (typically 30 μm).

In some examples, the spray device further comprises a cap, wherein the cap comprises cam tracks that engage with the drive member and the actuated valve, and wherein the drive member, the moveable sleeve and the cam tracks are arranged such that rotation of the cap causes: the actuated valve to move from the closed position to the open position; the drive member to withdraw, thereby priming the force generating means and simultaneously retracting the piston from the delivery chamber; and the actuated valve to move from the open position to the closed position.

In some examples, further rotation of the cap causes the piston to retract further from the delivery chamber after the actuated valve has been moved into the closed position, thereby causing air to be drawn into the delivery chamber through the one or more nozzles.

In another aspect of the disclosure, a method of operating a spray device is provided, the method comprising: opening the actuated valve; withdrawing the piston from the delivery chamber while the actuated valve is open, thereby allowing a portion of the liquid in the reservoir to flow into the delivery chamber through the actuated valve; closing the actuated valve; further withdrawing the piston while the actuated valve is closed, thereby causing air to flow into the delivery chamber though the one of more nozzles; and triggering the drive mechanism to force the piston into the delivery chamber, thereby causing the liquid in the delivery chamber to be driven through the one or more nozzles.

In another aspect of the invention, a spray device is provided for generating an aerosol, the spay device comprising; a pre-metered dose unit, the pre-metered dose unit comprising a flexible membrane and a rigid perforate element comprising one or more nozzles, the pre-metered dose unit being configured to hold a volume of a liquid; and a drive mechanism comprising a plunger configured to act on the flexible membrane of the unit dose container such that plunger forces the flexible membrane into the pre-metered dose unit in order to drive the fluid in the pre-metred dose unit through the one or more nozzles, thereby forming a liquid spray.

In some examples, the spray device further comprises: a collision means comprising at least one impaction surface located downstream of the one of more nozzles, such that, in use, the liquid that is driven through the one or more nozzles impacts the impaction surface of the collision means.

In some examples, the one or more nozzles comprise: at least two opposing nozzles, the opposing nozzles being positioned such that the projected areas of the holes at least partially intersect at the outlet side of the perforate element such that, in use, an aerosol is generated from at least two impinging jets formed when liquid is driven through the one or more nozzles.

In some examples, the spray device is configured such that the liquid that is driven through the nozzles forms a jet stream that breaks up into droplets by propagation of instability in the jet stream.

In some examples, wherein the drive mechanism comprises a spring loaded actuator which is loaded during a priming step and which has an acceleration gap between the triggering point and when it applies force to the plunger.

In some examples, the drive mechanism comprises compressed gas acting on a piston such that the piston has an acceleration gap between the triggering point and when it applies force to the plunger.

In some examples, each nozzle comprises an inlet and an outlet and each nozzle has a hydraulic diameter of 5 μm to 100 μm (typically 30 μm).

In some examples, each nozzle comprises an orifice that passes through the perforate element.

### BRIEF DESCRIPTION OF THE FIGURES

FIGS. 1 shows an embodiment of a rotary valve metering system in a first operating state.

FIG. 2 shows an embodiment of a rotary valve metering system in a second operating state.

FIG. 3 shows an axially actuated valve in a first operational state.

FIG. 4 shows an axially actuated valve in a second operational state.

FIG. 5 shows an axially actuated valve in a third operational state.

FIG. 6 shows an aerosolising means consisting of two nozzles directed such that the emitted jets impinge to form droplets.

FIG. 7 shows an aerosolising means consisting of consisting of a single nozzle that allows a single jet to be emitted.

FIG. 8 shows an alternative embodiment of an axially actuated valve.

FIG. 9 shows a unit dose implementation of dose metering.

FIG. 10 shows the unit dose device of FIG. 9 installed in a holder.

FIG. 11 shows the unit dose device of FIG. 10 after the liquid dose has been displaced by a piston.

FIG. 12 shows an implementation of a complete device that could be used with the different metering concepts and facilitates a fast start to the aerosolisation

### DETAILED DESCRIPTION OF THE FIGURES

The present disclosure relates to examples of spray devices including metering systems having actuated valves and spray devices that use pre-metred dose containers. The term actuated valve refers to valves that are actively moved between an open position and a closed position, as opposed to passive valves that are opened or closed as a result of a pressure differential. The valves may be actuated manually or may be actuated by a powered actuation mechanism. The actuation mechanism may be directly coupled to the valve, or may be connected indirectly, such as through a mechanical connection.

The spray devices described herein comprise nozzles through which liquids are ejected. The nozzles are typically an orifice that that passes through an end wall of the spray device. The end wall of the spray device is described here in as a perforate element, with the nozzles constituting the perforations in the element.

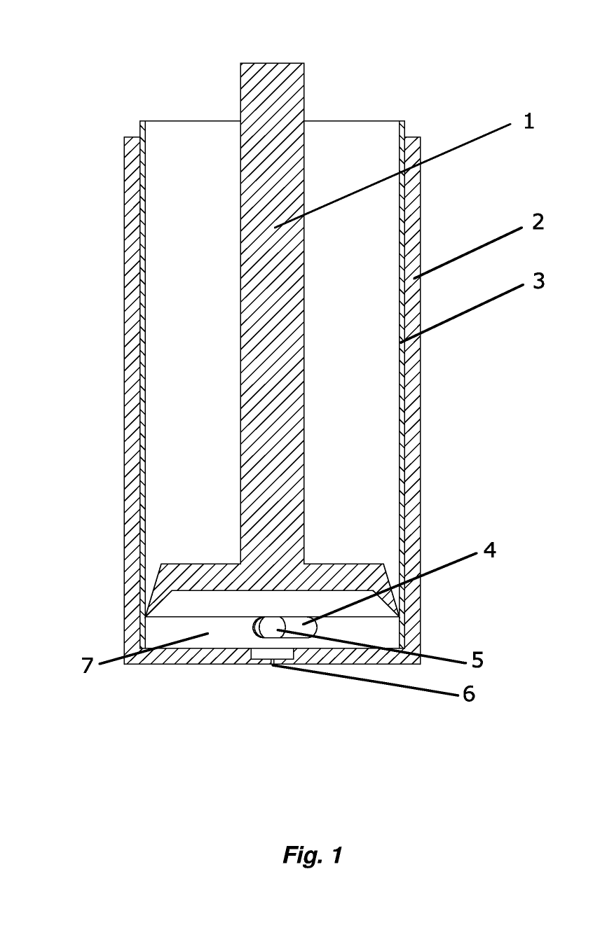

FIG. 1 shows a metering system for use in a spray device. The metering system comprises a cylindrical body wall 2, which is closed at one end. The closed end of the body wall comprises a nozzle 6. The nozzle 6 is a channel passing entirely through the body of the spray though which a fluid can be driven through under pressure. The nozzle 6 is typically 10 to 50 microns in diameter when used as part of an aerosolising means. In some embodiments the hydraulic radius of the nozzle is between 5 and 100 microns. Typically, the hydraulic radius will be around 30 microns.

A moveable sleeve 3 is disposed within the body wall 2. The sleeve 3 is also cylindrical and has approximately the same radius as the inner surface of the body wall 2. The sleeve fits closely to the inner surface of the surrounding body wall 2 and is able to rotate relative to the body wall 2. Thus, the moveable sleeve 3 and the body wall 2 form a double layered housing partically defining a delivery chamber 7 for holding a volume of liquid to be aerosolised.

A piston 1 is provided within the moveable sleeve 3 and, thus, also within the body wall 2. The piston 1 is able to rotate with the sleeve 3 and move axially relative to both the body wall 2 and sleeve 3. The piston 1 has an end section having the same cross section as the delivery chamber 7. The position of the piston end section defines the extent of the delivery chamber 7.

The piston 1 is shown with a moulded piston seal to help minimise the component count and assembly processes for cost reasons but this could be replaced by a separate seal such as an O-ring.

The moveable sleeve 3 comprises a sleeve fluid transfer port 4. The body wall 2 comprises a body fluid transfer port 5. The body wall and the sleeve wall cooperate to form an actuated valve system, the valve being open when the fluid transfer port 4 of the sleeve is aligned with the fluid transfer port of the body wall, thus providing a continuous path through which fluid can flow from outside the delivery chamber into the delivery chamber. Conversely, the actuated valve is closed when the fluid transfer ports do not overlap, because the path through the body wall fluid transfer port 4 is blocked by the moveable sleeve.

In some embodiments, the metering system is disposed within a liquid reservoir. In the position shown in FIG. 1, the sleeve fluid transfer port 4 in the sleeve 3 is aligned with the body fluid transfer port 5 in the body wall 2. When the sleeve port 4 and the body port 5 are aligned, it is possible for liquid to flow, via the two fluid transfer ports, from the reservoir outside of the body wall 2 into the delivery chamber 7 inside the sleeve 3. The sleeve 3 and the body 2, in this case, act as an actuated valve that is controlled to open or close a channel between a reservoir and the delivery chamber 7 by rotating the sleeve 3 relative to the spray body 2.

As the piston 1 is withdrawn, the delivery chamber 7 is filled by liquid drawn from a reservoir through the aligned fluid transfer ports 4 and 5.

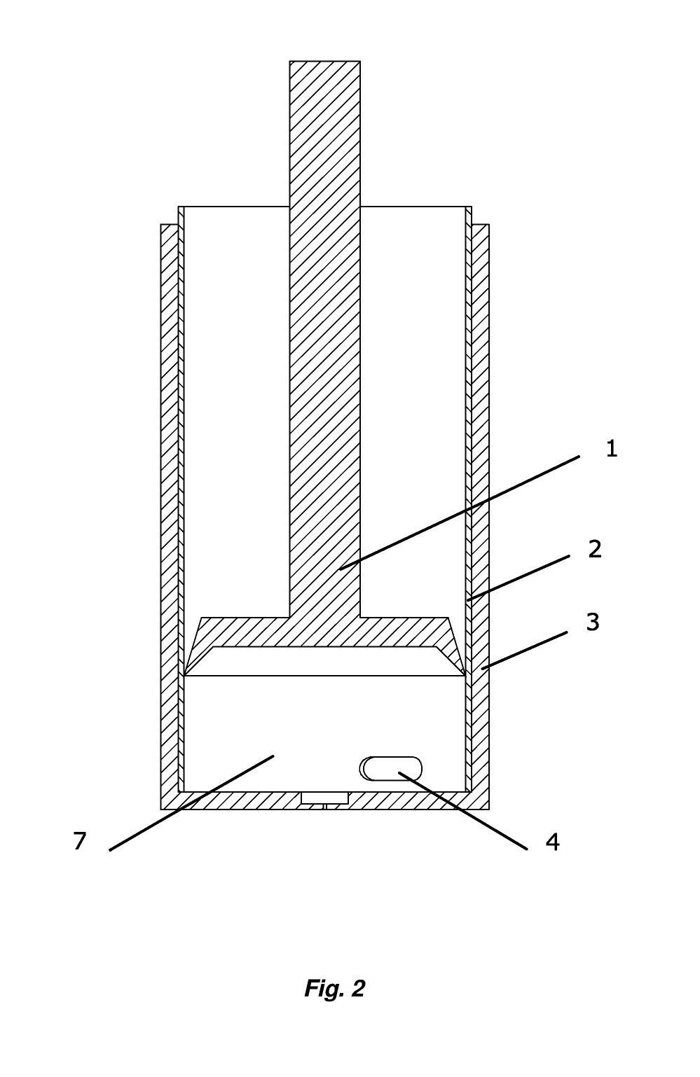

FIG. 2 shows the metering system in a second position, in which the piston 1 has been withdrawn to a predetermined set-point. As the piston 1 is withdrawn, liquid from the reservoir flows through the open valve (i.e. though the aligned fluid transfer ports) into the delivery chamber 7. Prior to the piston being driven back into the delivery chamber 7, the actuated valve is closed by rotating the piston 1 and sleeve 2 relative to the body wall 2 such that the sleeve fluid transfer port 4 no longer aligns with the body wall fluid transfer port 5.

In some examples, the actuated valve is closed before the piston 1 has been fully withdrawn, in order to draw a small amount of air into the delivery chamber 7 through the ejection nozzles.

In some examples, the one or both of the piston 1 and the moveable sleeve 3 is connected to a cap that can be rotated relative to the body wall 2. In this case the actuation of the valve occurs through a rotation of the cap. The cap could be rotated using a motor or manually by a user. In some examples, the piston 1 or the sleeve 3 is directly rotated using a motor.

After the closing of the actuated valve, the delivery chamber 7 is sealed other than the fluid path through the one of more nozzles. The piston is then driven into the delivery chamber, which generates the pressure needed for the formation of an aerosol.

In some examples, the force generating means that causes the piston 1 to be driven into the delivery chamber 7 comprises a spring, which is primed as the piston is withdrawn. In some examples, the piston 1 is driven into the delivery chamber 7 using a compressed gas, which is expanded into an expansion chamber located above the piston 1.

In some examples, a collision means comprising at least one impaction surface is located downstream of the one of more nozzles, such that, in use, the liquid that is driven through the one or more nozzles impacts the impaction surface of the collision means in order to assist in the aerosolising process.

In some examples, the ejection parameters are configured such that the liquid that is driven through the nozzles forms a jet stream that breaks up into droplets by propagation of instability in the jet stream.

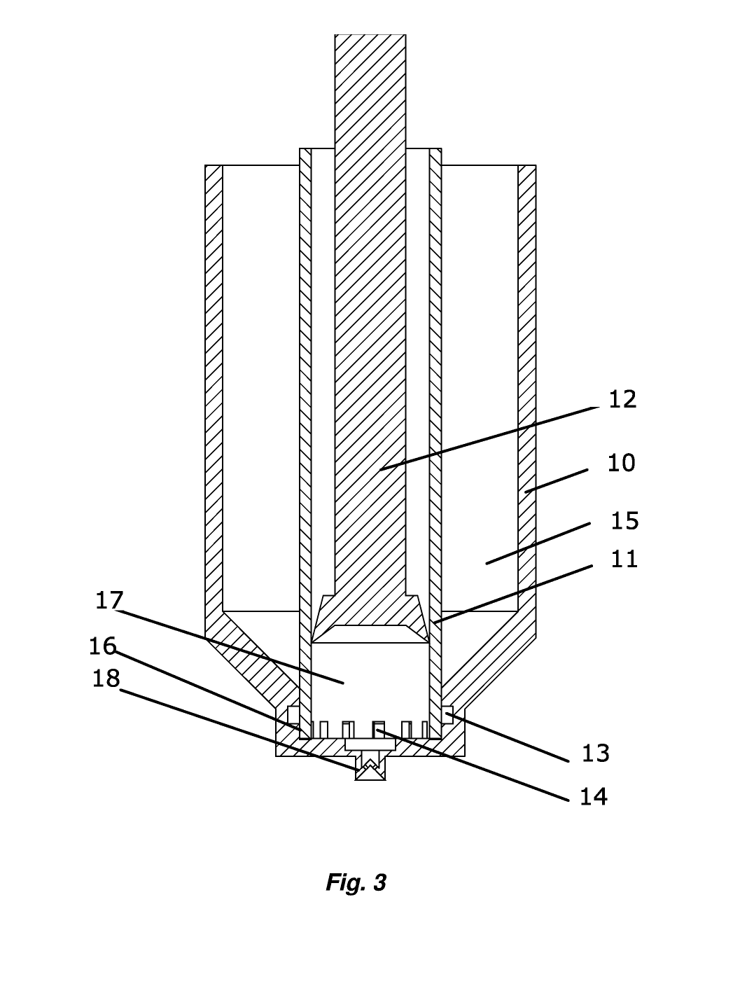

FIG. 3 shows embodiment spray device, in which the actuated valve is formed by a moveable sleeve 11 fitting into a recess 16 defined by the outer body 10 of a reservoir 15. A piston 12 disposed within the sleeve 11 defines the extent of a delivery chamber 17 within the sleeve 11.

The spray device comprises an outer body 10 having a narrow first portion defining a reservoir 10 and a wide second portion defining a recess 16. The moveable sleeve 11 is disposed within the outer body 10, with a portion of the sleeve 11 being disposed within the recess 16 and a portion of the sleeve 11 being disposed in the reservoir 15. The inner radius of the first portion is approximately the same as the outer radius of the sleeve 11, so that the sleeve 11 fits closely to the first portion of the outer body 10 when positioned in the recess. The space between the sleeve 11 and the wide second portion defines a reservoir 15 that can be filled with a liquid. The close fit between the sleeve and the narrow first portion prevents the liquid from entering the recess from the reservoir. To further ensure that liquid does not enter the reservoir, an elastomeric O-ring seal is provided in an O-ring slot 13 in the narrow first portion. This forms a seal between the narrow first portion of the outer body 10 and the moveable sleeve 11, which prevents the liquid from flowing into the recess around the outside of the moveable sleeve 11.

In some examples, the elastomeric O-ring and the O-ring slot 13 are omitted and a seal is formed as a result of the contact between the moveable sleeve 11 and narrow outer body of the recess 16. This is particularly effective as pressure within the moveable sleeve 11 will act to expand the sleeve 11 against the walls of the recess 16.

In this example the end of the sleeve 11 has one or more fluid transfer ports 14 in at one end. In the shown example, the fluid transfer ports 14 are slots that extend from the end of the sleeve 11 some distance up the side of the sleeve 11. When the moveable sleeve is fully 11 inserted into the recess, as shown in FIG. 3, the fluid transfer ports 14 in the sleeve are entirely covered by the outer body 10 in the recess 16. In this position, the actuated valve formed by the moveable sleeve 11 and the outer body 10 is closed.

When the sleeve 11 is partially, or fully, withdrawn from the recess 16, the fluid transfer ports become partially or fully exposed to the reservoir 15. This creates a fluid path between the fluid volume in the fluid reservoir 15 in the body 10 to the delivery chamber 17 via the fluid transfer ports in the sleeve 14. In this position, the actuated valve is open.

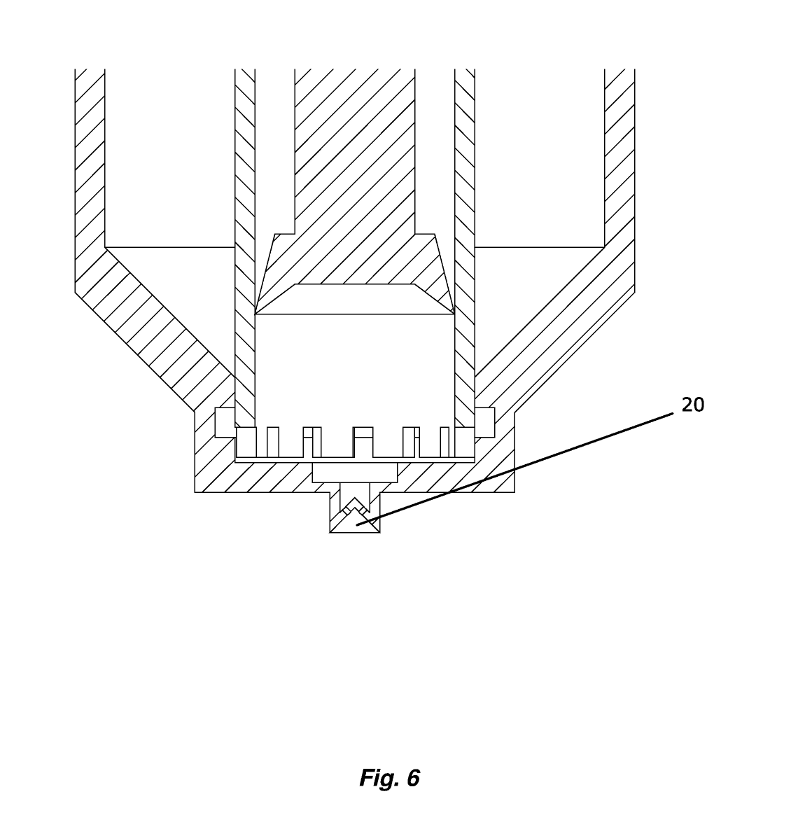

In the examples in which the fluid transfer ports 14 are elongated slots, the actuated valve can be opened without fully withdrawing the sleeve 11 from the recess 16. This is advantageous in terms of maintaining alignment between these two components. The spray device has a nozzle arrangement 18 comprising two nozzles that are directed towards one another such that the ejected streams collide outside of the delivery chamber 17. The nozzle arrangement 18 forming part of the aerosolising means is more fully described with reference to FIG. 6.

In some examples, the sleeve 11 is moved using a motor. In other examples, the sleeve 11 is moved manually.

In some examples, the sleeve 11 comprises a flanged region that engages cam tracks in a rotatable cap. In such an example, an axial motion of the sleeve can be brought about by a rotation of the cap.

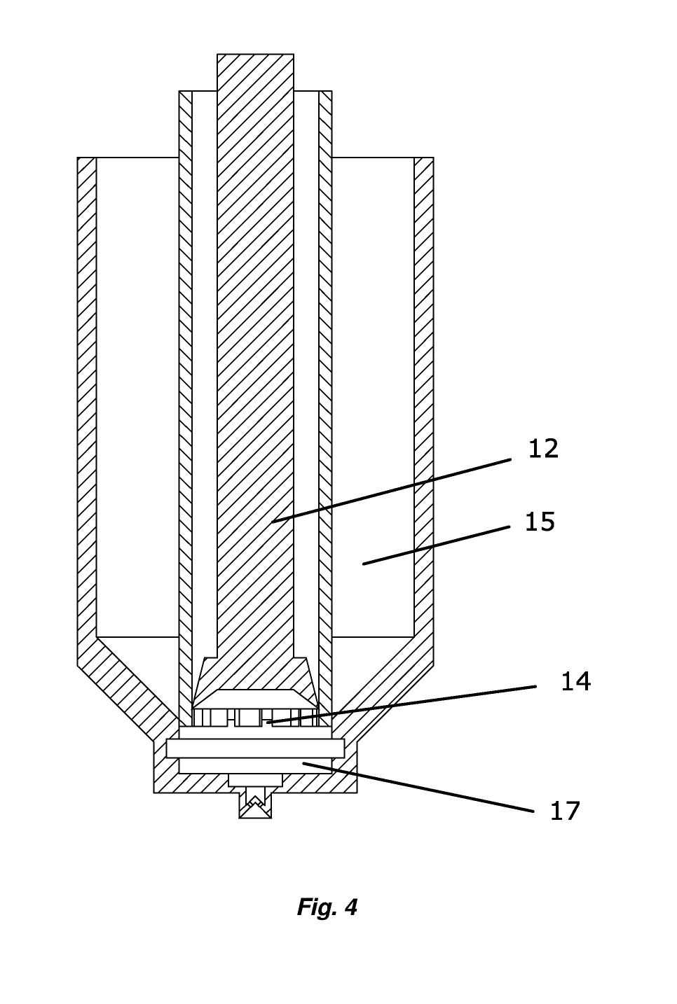

FIG. 4 shows the spray device of FIG. 3 where the moveable sleeve 11 is partially withdrawn from the recess 16, opening the valve and creating a fluid communication path between the fluid reservoir 15 through the slots 14 into the delivery chamber 17. The piston 12 can be withdrawn, causing liquid in the reservoir to be drawn into the delivery chamber 17 through the slots 14. This process allows the delivery chamber 17 to fill prior to an ejection.

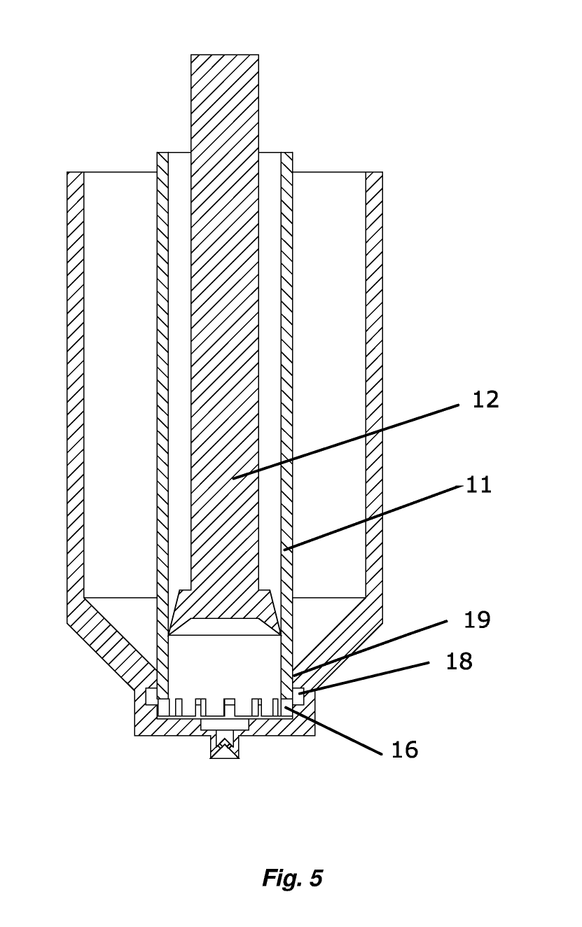

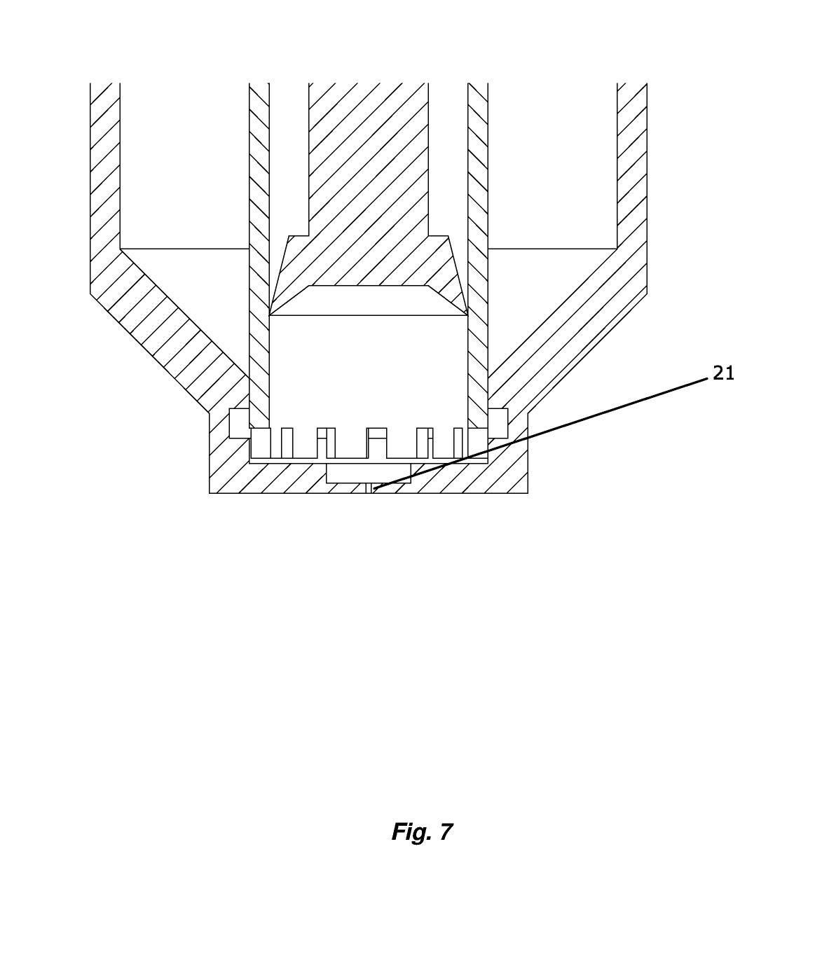

FIG. 5 shows the device from FIG. 3 in a position where the sleeve 11 has been returned to full engagement with the recess 16, thus closing the actuated valve. The delivery chamber is sealed both through the O-ring situated in the O-ring slot 18 and as a result of the contact seal 19 formed between the sleeve 11 and the outer body wall 10. This position occurs after the delivery chamber 17 has been filled, as described with reference to FIG. 4. In this position, the spray device is ready for ejection and, when the piston 12 moves to reduce the volume of the delivery chamber 17 pressure is generated to force liquid through the delivery nozzles of the aerosol means. The aerosol means are shown in more detail in FIGS. 6 and 7.

FIG. 6 shows an aerosolising means comprising two nozzles directed towards one another, such that the emitted jets impinge to form droplets. In the shown examples, each of the nozzles has an axis oriented at an angle of between 55 and 125 degrees (preferably 90 degrees) to an external surface of the thin walled section, the opposing nozzles being positioned such that the projected areas of the holes at least partially intersect at the outlet side of the nozzle. This causes the two ejected streams to collide in a region 20 outside of the delivery chamber 17.

In some examples, the aerosolising means has more than two nozzles.

FIG. 7 shows an aerosolising means comprising a perforate element having a single nozzle 21 that allows a single jet to be emitted. The nozzle 21 is configured such that the liquid that is driven through the nozzle 21 forms a jet stream that breaks up into droplets by propagation of instability in the jet stream.

In some embodiments, a collision means comprising at least one impaction surface is located downstream of the nozzle 21, such that, in use, the liquid that is driven through the nozzle 21 impacts the impaction surface of the collision means.

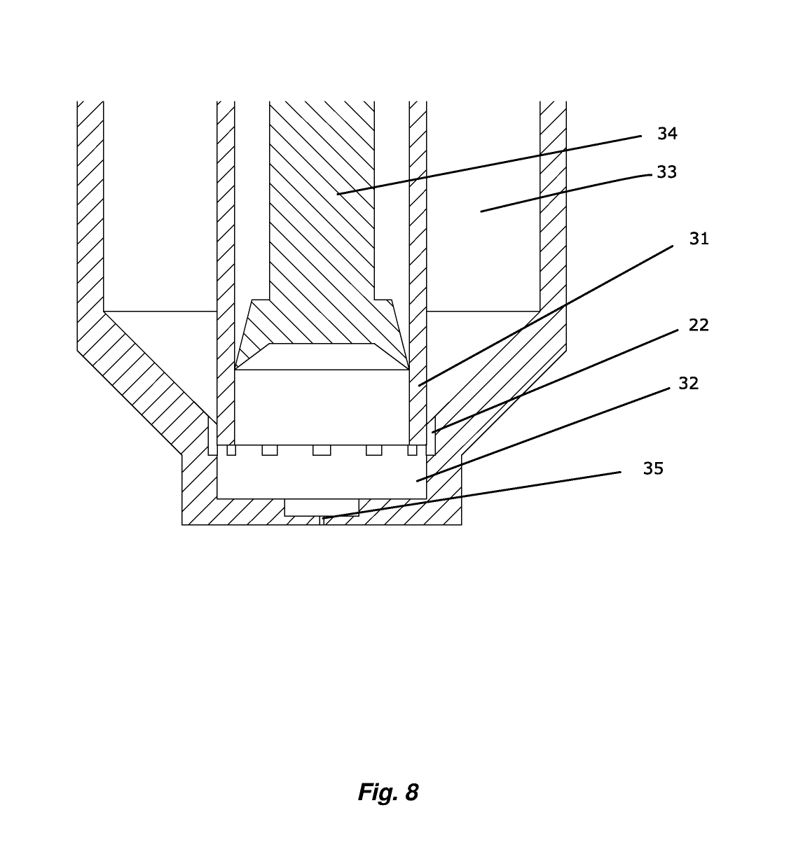

FIG. 8 shows an alternative example of a spray device comprising an actuated valve. This spray device is similar in structure to the device shown in FIG. 3. In this example, however, the moveable sleeve 31 does not comprise fluid transfer ports. Instead, a plurality of slots/channels 22 is incorporated into the narrow section of the outer body 32. The plurality of slots 22 extend down from the wide section of the outer body and radially into the narrow section of the outer body. Thus, when the sleeve 31 is fully inserted into the recess 32, the slots remain in fluid communication with the reservoir 33. As the sleeve is axially withdrawn from the recess, the slots 22 are partially exposed to the recess. This creates a fluid path between the fluid reservoir 33 and the delivery chamber. When the 31 sleeve is fully inserted into the recess, the actuated valve is closed. When the sleeve 31 is withdrawn to expose the slots to the delivery chamber 32, the valve is open.

When the piston 34 is withdrawn fluid can flow from the reservoir 33 to the chamber 32. When the piston is fully withdrawn then the sleeve 31 is returned to full engagement with the chamber 32 to reform the sealed chamber in preparation for the advancing of the piston to eject fluid through the nozzles of the aerosolising means 35.

In each of the embodiments in FIGS. 1 to 8 the provision of an actuated valve allows the timing of the opening and closing of the valve between the delivery chamber and liquid reservoir can be tuned for optimum effect. This allows the valve opening to be delayed or advanced with respect to the movement of the piston 1 in comparison with passive valve mechanisms.

For example, if during the filling process the actuated valve is closed before the piston 1 is fully withdrawn, a small dose of air can be drawn into the delivery chamber 17 through the one or more nozzles 6. This is particularly advantageous when working with shear thinning fluids, because the ejection process can begin by ejecting air, during which time the liquid can be accelerated; this causes the liquid to shear thin before being introduced to the nozzle, producing a reliable ejection.

Similarly, active control of the movement of the valve allows closure of the valve can be ensured before fluid dispensing is started. This overcomes the challenges seen with passive non-return valves that are otherwise used. Typically non-return valves designed for low pressure bias operation need some counter flow to generate a pressure differential to close. This slows down the closure and can lead to inconsistency to the start of the pressurisation of the chamber and leakage from the chamber.

Alternatively, biased sprung loaded non-return valves are be used; however these have the opposite issue of requiring a pressure differential to be opened, and so at the start of the filling event an negative pressure is needed in the delivery chamber. This has potential negative consequences: negative pressure can lead to out gassing of the fluid which will adversely affect metering accuracy and add to the compliance in the delivery chamber at the start of the pressurisation and spray event

Furthermore, in many spray devices the delivery nozzle is open to the atmosphere; applying a negative pressure to the metering chamber can, therefore, draw unintended air into the metering chamber through the nozzle, thus further compromising metering accuracy and consistency and increasing compliance due to the compressibility of the air within the chamber. By providing an actuated valve that can be controlled to open or close as a depending on the position of the piston, it is possible to eliminate the above issues and provide an aerosol spray device which can control the fluid and air flow into the chamber.

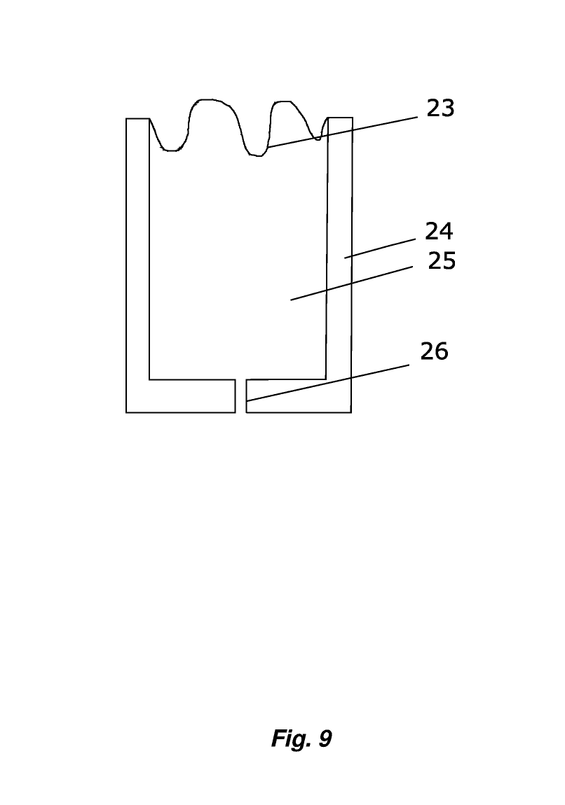

FIG. 9 shows an example of a pre-metered unit dose which simplifies the operation of aerosol devices by eliminating valving and supply metering functions. The pre-metred unit dose container comprises a rigid outer body 24 defining a delivery chamber 25 and comprising an ejection nozzle 26. The delivery chamber is pre-supplied with a liquid to be aerosolised. The unit dose container further comprises a flexible membrane 23 on one surface, which can be compressed with a piston in order to drive a fluid from the delivery chamber 25 through the nozzle 26. The pre-metered unit dose container is inserted prior to use into a spray device having a piston and drive mechanism for ejecting the liquid from the container.

The main consideration regarding the achievement of a reliable fast start up when using a pre-metered unit dose is the application of the required force to the unit dose and the displacement between the drive mechanism and the unit dose once a unit dose is loaded into a spray device.

The provision of an enclosed dose contained allows the use of a drive mechanism with an acceleration gap to provide a fast start to the delivery event. By using a physical travel stop to limit the extent of motion of the drive mechanism or piston, a fast end to the pressurisation can also be achieved.

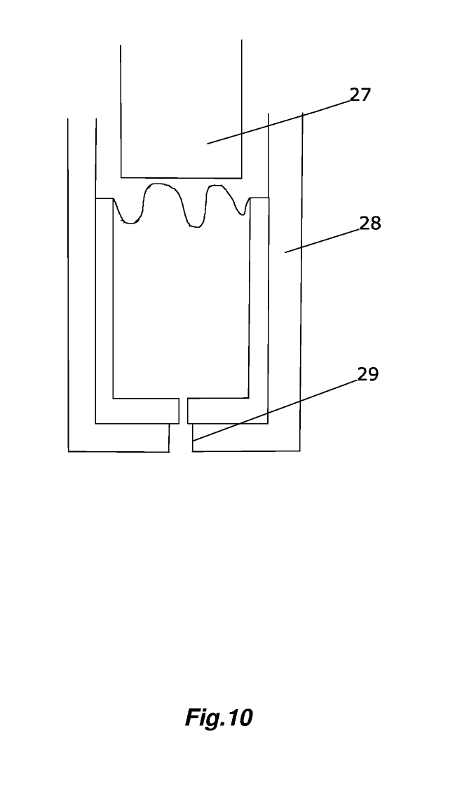

FIG. 10 shows the unit dose device installed in a holder 28 which provides additional support for the capsule during dispensing. The holder 28 has an opening 29 for allowing ejected liquid to escape from the unit dose device. The plunger/piston 27 is shown in position ready to apply load to the membrane to start dispensing of the liquid. A further advantage of this approach is the elimination of sliding seals for a piston 1 by the substitution of the flexible membrane. This has advantages in terms of loading the capsule and ensuring a leak free function.

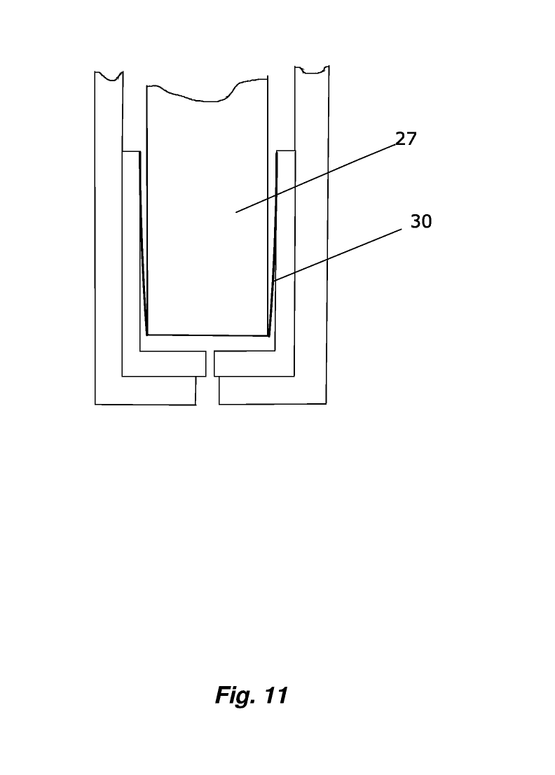

FIG. 11 shows the unit dose capsule once the plunger 27 has displaced the flexible membrane, shown in displaced position 30 and delivered the liquid dose at high pressure through the nozzles of the aerosolising means.

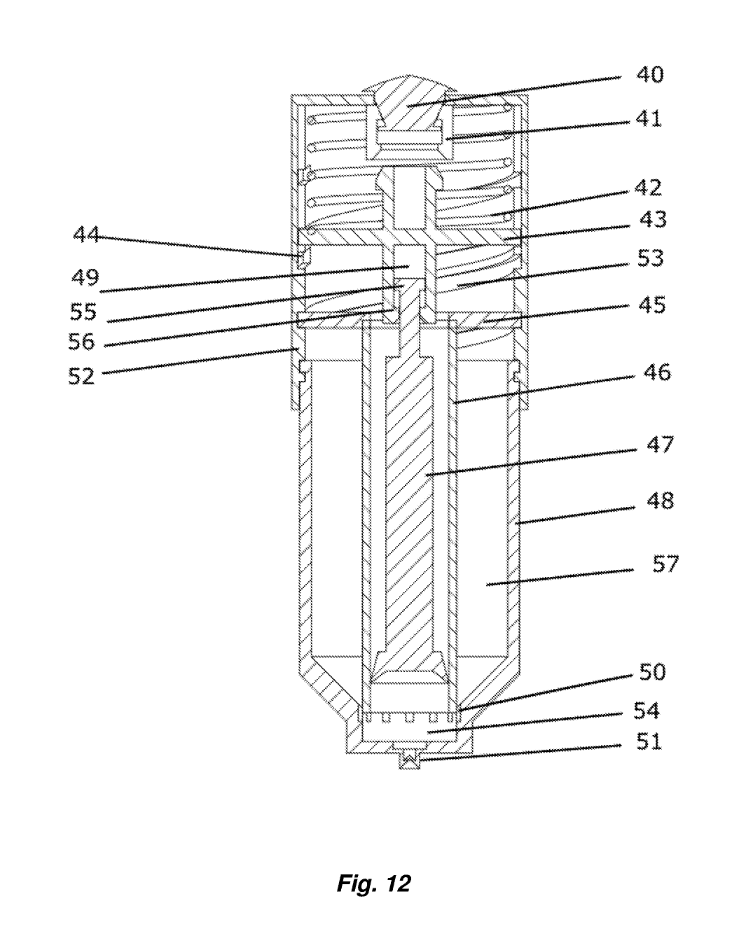

FIG. 12 shows an embodiment of a spray device in the actuation of the actuated valve is linked to the withdrawal of a piston 47 and the priming of a drive mechanism 43 through the use of cam tracks (53 and 44). The spray device incorporates an actuated valve comprising a sleeve 46 (in the example shown in FIG. 12, the actuated valve is of the type described with reference to FIG. 8, though any of the types of actuated valve described in this specification may be use), a spring loaded drive member 43, an actuation trigger 40, and an acceleration gap 49 for between the piston 47 and spring loaded drive member 43. In this example, the drive force is generated using a spring 42 coupled to the drive member 43. The drive member 43 is used both to withdraw the piston 47 from the delivery chamber 54 and to drive the piston 47 back into the delivery chamber 54.

The device further comprises a cap 52 having cam tracks 53 that engage the sleeve 46 and the drive mechanism 43. The axial position of the sleeve 46 is determined by the rotation of the cap 52, as is the priming of the drive member 43 and the withdrawal of the piston 47. This allows the relative timing of the valve opening and closing to be carefully co-ordinated with the priming and the piston withdrawal.

Specifically, as the cap 52 is rotated relative to the body 48, the spring carrier 43 is moved by the cam track 44 such that it compresses the spring 42. As the cap 52 is rotated it also withdraws the sleeve 46 through the interaction of the disc 45 attached to the sleeve 46 running in cam tracks 53.

The profile of the cam tracks 53 are arranged to control the motion of the sleeve 46 to operate the actuated valve functions created by the interaction of the sleeve 46 and the slots 50 in the delivery chamber 54.

The piston is coupled to the spring carrier 43 through a coupling mechanism 56 which latches on to the head 55 of a piston rod forming part of the piston 47. The coupling mechanism 56 has a degree of play, which allows the piston to move a pre-defined distance independently of the spring carrier 43. As the piston 47 is lifted by the drive member 43 via the coupling mechanism 56 acting on the head of the piston rod 55, the sleeve 46 is also raised through interaction with the cam tracks in the cap 52 and the top of the sleeve 45 such that the valve is opened allowing fluid to flow from the reservoir 57 into the chamber 54 through now open fluid path via the slots 50. As the rotation of the cap 52 is continued, the drive member 43 is latched into a latch 41 within the cap 52. The cam track 53 for the sleeve 47 is arranged such that, as the rotation of the cap 52 continues, the sleeve 47 is re-inserted back into full engagement with the recess to form a sealed delivery chamber 54.

The spring carrier 43 is disengaged from the cam track in the cap 52 by running into an axial track that provides clearance for the drive member 43. The play between the drive member 43 and the piston 47 caused by the coupling mechanism creates a gap between the drive member 43 and the piston 47. When the actuation button 40 is pressed, the latch is released and the spring carrier drive member 43 is accelerated back towards the piston until the gap 49 is taken up. This acceleration space allows for a fast rise in pressure within the chamber 54 which is beneficial for clean start-up of the aerosol.

The cam tracks 44 that drive the axial motion of the spring carrier 43 and the tracks 53 that drive the axial position of the sleeve 46 can be arranged to set the timing of the valve opening such that the valve opens when the piston starts to withdraw to allow dose metering and closes as the spring carrier 43 engages with the trigger 41 such that, once triggered, the pressure in the dose chamber rises appropriately.

In a similar example, the rotary valve arrangement described in conjunction with FIGS. 1 and 2 is used instead of the axial vale arrangement. In this example, the valve can be momentarily opened by the action of the drive member 43 rotating and applying a small rotation to the sleeve 3 relative to the body 2 towards the end of the dose delivery. This vents any residual pressure from the delivery chamber 7.

In another example, using the axially actuated valve illustrated in FIG. 12, the drive member 43 can press the sleeve further into engagement with the delivery chamber and so open a discharge port to waste to end the delivery abruptly.

Similar functionality is provided by the axial track in the cap that provides clearance for the spring carrier if the track is curved towards the end of travel. This induces rotary motion in the spring carrier relative to the cap and in turn this can release the cap from its initial engagement with the body 48 and so releasing the residual spring load and so rapidly reducing the delivery pressure and giving a clean end to the delivery.

1. A spray device for generating an aerosol, the spray device comprising:
a reservoir for containing a store of a liquid;
a delivery chamber for containing a dose of the liquid received from the reservoir prior to ejection;
a perforate element comprising one or more nozzles;
a drive mechanism configured to drive liquid from the delivery chamber through the one or more nozzles, thereby forming a liquid spray having one or more streams of liquid; and
a fluid metering system, the fluid metering system comprising an actuated valve that is actuatable between a closed position and an open position; wherein
the reservoir and the delivery chamber are in fluid communication when the actuated valve is open, and the reservoir and the delivery chamber are not in fluid communication when the actuated valve is closed, and wherein the opening and closing of the actuated valve can be sequenced to control the flow of fluid from the reservoir to the delivery chamber.

2. The spray device of claim 1, wherein the actuated valve comprises a moveable sleeve.

3. The spray device of claim 2, wherein the actuated valve comprises a body wall disposed between the reservoir and the delivery chamber.

4. The spray device of claim 3, wherein the moveable sleeve is disposed between the body wall and the delivery chamber.

5. The spray device of claim 4, wherein the moveable sleeve is moveable with respect to the body wall.

6. The spray device of claim 5, wherein the actuated valve is moveable between the open position and the closed position by a rotary motion of the moveable sleeve.

7. The spray device of claim 5, wherein the actuated valve is moveable between the open position and the closed position by an axial translation of the moveable sleeve.

8. The spray device of claim 2, wherein the moveable sleeve comprises at least one fluid transfer port.

9. The spray device of claim 3, wherein the body wall comprises at least one fluid transfer port.

10. The spray device of claim 9, wherein the actuated valve is in the open position when at least one fluid transfer port of the body wall is aligned with at least one fluid transfer port of the moveable sleeve.

11. The spray device of claim 2, wherein the reservoir comprises an outer body, the outer body comprising:
a first portion having approximately the same radius as the moveable sleeve, and
a second portion having a greater radius than the moveable sleeve, and wherein
the moveable sleeve is disposed within the reservoir with a portion of the moveable sleeve being disposed within the first portion of the outer body, such that, in use, the liquid in the reservoir is held in contact with the second portion of the outer body and a portion of the sleeve that is not disposed within the first portion.

12. The spray device of claim 11, wherein
the moveable sleeve comprises at least one fluid transfer port; and wherein
the actuated valve is closed when the moveable sleeve is in in a first position and wherein the actuated valve is open when the moveable sleeve is in a second position, wherein:
in the first position the fluid transfer port is positioned adjacent to the first portion of the outer body, and
in the second position, the fluid transfer port is positioned such that it is surrounded by the second portion of the outer body,
such that, in use, the liquid in the reservoir is prevented from passing from the into the delivery chamber when the moveable sleeve is in the first position, and the liquid in the reservoir is able to pass from the reservoir into delivery chamber through the at least one fluid transfer port when the moveable sleeve is in the second position.

13. The spray device of claim 11, wherein
the first portion outer body comprises a plurality of channels that extend from the reservoir into the first portion, and wherein
the actuated valve is closed when the moveable sleeve is in a first position wherein the channels are covered by the moveable sleeve, and
the actuated valve is open when the moveable sleeve is in a second position wherein the channels are partially uncovered by moveable sleeve, with the channels forming a path between the reservoir and the delivery chamber.

14. The spray device of claim 12, further comprising: a seal disposed between the first portion of the outer body and the moveable sleeve for preventing the liquid in the reservoir from flowing between the first portion of the outer body and the moveable sleeve.

15. The spray device of claim 1, further comprising:
a collision means comprising at least one impaction surface located downstream of the one of more nozzles, such that, in use, the liquid that is driven through the one or more nozzles impacts the impaction surface of the collision means.

16. The spray device of claim 1, wherein the one or more nozzles comprise:
at least two opposing nozzles, the opposing nozzles being positioned such that projected areas of the holes at least partially intersect at the outlet side of the perforate element such that, in use, an aerosol is generated from at least two impinging jets formed when liquid is driven through the one or more nozzles.

17. The spray device of claim 2, configured such that the liquid that is driven through the nozzles forms a jet stream that breaks up into droplets by propagation of instability in the jet stream.

18. The spray device of claim 17, wherein the drive mechanism comprises a piston.

19. The spray device of claim 18, wherein the drive mechanism further, comprises a drive member, the drive member being coupled to a force generating means, wherein the spray device is configured such that upon triggering the drive mechanism, the force generating means accelerates the drive member across an acceleration gap before engaging the piston, after which the drive mechanism continues to drive the piston into the delivery chamber in order to drive the liquid from the delivery chamber through the one or more nozzles.

20. The spray device of claim 19, wherein:
the force generating means comprises a spring.

21. The spray device of claim 18, wherein the force generating means comprises a compressed gas.

22. The spray device of claim 1, further comprising: an actuator configured to move the actuated valve between the open position and the closed position.

23. The spray device of claim 1, wherein each nozzle comprises an inlet with a hydraulic diameter of 5 μm to 100 μm (typically 30 μm) and an outlet with a hydraulic diameter of 5 μm to 100 μm (typically 30 μm).

24. The spray device of claim 19, further comprising a cap, wherein the cap comprises cam tracks that engage with the drive member and the actuated valve, and wherein the drive member, the moveable sleeve and the cam tracks are arranged such that rotation of the cap causes:
the actuated valve to move from the closed position to the open position;
the drive member to withdraw, thereby priming the force generating means and simultaneously retracting the piston from the delivery chamber; and
the actuated valve to move from the open position to the closed position.

25. The spray device of claim 24, wherein:
further rotation of the cap causes the piston to retract further from the delivery chamber after the actuated valve has been moved into the closed position, thereby causing air to be drawn into the delivery chamber through the one or more nozzles.

26. A method of operating the spray device of claim 19, comprising:
opening the actuated valve;
withdrawing the piston from the delivery chamber while the actuated valve is open, thereby allowing a portion of the liquid in the reservoir to flow into the delivery chamber through the actuated valve;
closing the actuated valve;
further withdrawing the piston while the actuated valve is closed, thereby causing air to flow into the delivery chamber though the one of more nozzles; and
triggering the drive mechanism to force the piston into the delivery chamber,
thereby causing the liquid in the delivery chamber to be driven through the one or more nozzles.

27. A spray device for generating an aerosol, the spray device comprising;
a pre-metered dose unit, the pre-metered dose unit comprising a flexible membrane and a rigid perforate element comprising one or more nozzles, the pre-metered dose unit being configured to hold a volume of a liquid; and
a drive mechanism comprising a plunger configured to act on the flexible membrane of the unit dose container such that plunger forces the flexible membrane into the pre-metered dose unit in order to drive the fluid in the pre-metred dose unit through the one or more nozzles, thereby forming a liquid spray.

28. The spray device of claim 27, further comprising:
a collision means comprising at least one impaction surface located downstream of the one of more nozzles, such that, in use, the liquid that is driven through the one or more nozzles impacts the impaction surface of the collision means.

29. The spray device of claim 28, wherein the one or more nozzles comprise:
at least two opposing nozzles, the opposing nozzles being positioned such that projected areas of the holes at least partially intersect at the outlet side of the perforate element such that, in use, an aerosol is generated from at least two impinging jets formed when liquid is driven through the one or more nozzles.

30. The spray device of any of claim 29, configured such that the liquid that is driven through the nozzles forms a jet stream that breaks up into droplets by propagation of instability in the jet stream.

31. The spray device of claim 30, wherein the drive mechanism comprises a spring loaded actuator which is loaded during a priming step and which has an acceleration gap between the triggering point and when it applies force to the plunger.

32. The spray device of claim 31, wherein the drive mechanism comprises compressed gas acting on a piston such that the piston has an acceleration gap between the triggering point and when it applies force to the plunger.

33. The spray device of claim 32, wherein each nozzle comprises an inlet and an outlet and each nozzle has a hydraulic diameter of 5 μm to 100 μm (typically 30 μm).

34. The spray device of claim 27, wherein each nozzle comprises an orifice that passes through the perforate element.
